(19) United States
(12) Patent Application Publication
(10) Pub. No.: US 2023/0321404 A1
(43) Pub. Date: Oct. 12, 2023

(54) CATHETER ARRANGEMENT INCLUDING A VALVE ELEMENT ELASTICALLY DEFORMABLE BY FLUID PRESSURE

(71) Applicant: B. Braun Melsungen AG, Melsungen (DE)

(72) Inventors: Stefan Klaas, Ludwigsau (DE); Uwe Erik Schneider, Spangenberg (DE); Kavintharan Pala Sandaram, Bayan Lepas, Penang (MY); Wei Jin Soon, Hilir Sungai Pinang, Penang (MY); Aik Aun Tan, Solok Thean Teik, Penang (MY); Jeremy Goh Tee Liang, Bayan Lepas, P.Pinang (MY); Hui Kuun Teoh, Bayan Lepas, Penang (MY)

(21) Appl. No.: 18/019,777

(22) PCT Filed: Aug. 4, 2021
(86) PCT No.: PCT/EP2021/071797

§ 371 Date: Feb. 3, 2023

(30) Foreign Application Priority Data
Aug. 6, 2020 (DE) 10 2020 209 931.6

(51) Int. Cl. A61M 25/06; A61M 39/24; A61M 5/158

(52) U.S. Cl. CPC A61M 25/0625; A61M 39/24; A61M 2039/242; A61M 2039/2433; A61M 5/158

(57) ABSTRACT

A catheter arrangement and use of a catheter arrangement in infusion therapy. The catheter arrangement includes a catheter with a housing body, a hollow needle, a tube fitted onto a distal end of the housing body, a valve element arranged in the housing body with a fluid passage, and a fluid-conducting path extending through the housing body, the fluid passage and the tube. The needle extends longitudinally through the fluid-conducting path in a state of readiness of the catheter arrangement, and is drawn out of the fluid-conducting path in a state of use of the catheter arrangement. The valve element has an elastic wall portion through which the fluid passage extends. The fluid passage can be shifted by a fluid-pressure-induced elastic deformation of the wall portion between an open state, in which the fluid passage is open, and a closed state, in which the fluid passage is closed.

### CROSS-REFERENCE TO RELATED APPLICATIONS

This application is the United States national stage entry of International Application No. PCT/EP2021/071797, filed Aug. 4, 2021, and claims priority to German Application No. 10 2020 209 931.6, filed Aug. 6, 2020. The contents of International Application No. PCT/EP2021/071797 and German Application No. 10 2020 209 931.6 are incorporated by reference herein in their entireties.

### FIELD

The invention relates to a catheter arrangement, comprising a catheter, which has a hollow housing body, a tube element fitted onto a distal end of the housing body, a valve element which is arranged in the housing body and is provided with a fluid passage, and a fluid-conducting path which is made to extend longitudinally through the housing body, the fluid passage and the tube element between a proximal inlet side and a distal outlet side, and comprising a hollow needle which, in a state of readiness of the catheter arrangement, is made to extend longitudinally through the fluid-conducting path, and which, in a state of use of the catheter arrangement, is drawn out of the fluid-conducting path in the proximal direction.

### BACKGROUND

A catheter arrangement of this type is known from EP 1 911 485 B1 and is provided for use in infusion therapy. According to customary medical parlance, the known catheter arrangement may also be referred to as an indwelling venous catheter. The known catheter arrangement has a hollow housing body in the form of a hollow-cylindrical catheter bushing, to the distal end of which a tube element, which is referred to as a catheter, is attached. A valve element with a fluid passage is arranged in the hollow-cylindrical catheter bushing. A fluid-conducting path is made to extend longitudinally through the hollow-cylindrical catheter bushing, the fluid passage and the tube element between a proximal inlet side and a distal outlet side. In addition, the known catheter arrangement has a hollow needle which is attached to a needle bushing. In a state of readiness of the catheter arrangement, said hollow needle is made to extend longitudinally in the distal direction and through the catheter bushing, the fluid passage in the valve element and through the tube element starting from the proximal inlet side in the direction of the distal outlet side. In a state of use of the catheter arrangement, the hollow needle is drawn out of the fluid-conducting path in the proximal direction. In the case of the known catheter arrangement, the valve element is configured in the form of a flat disk. In order to actuate the valve element, a valve actuation element is arranged in the catheter bushing and can be shifted in the axial direction relative to the valve element. The valve actuation element interacts with a fluid-conducting component which can be connected proximally to the catheter bushing. In a connected state of the fluid-conducting component, the valve actuation element is shifted in the distal direction by mechanical contact connection of the valve element, as a result of which the fluid passage is opened. When the fluid-conducting component is removed, the valve actuation element is shifted in the proximal direction, contact with the valve element is ceased and the fluid passage is thereby closed.

### SUMMARY

It is the object of the invention to provide a catheter arrangement of the type mentioned at the beginning which has a simple design and permits improved use safety in comparison to the prior art.

This object is achieved in that the valve element has an elastic wall portion through which the fluid passage is made to extend, and in that the wall portion is elastically deformable under the action of a fluid pressure, wherein the fluid passage—in the state of use of the catheter arrangement—can be shifted by means of a fluid-pressure-induced elastic deformation of the wall portion between an open state, in which the fluid passage is open, and a closed state, in which the fluid passage is closed. The solution according to the invention makes it possible in particular to dispense with a valve actuation element which is arranged in the housing body, for shifting the fluid passage between the open position and the closed position. This is because, instead of a customary actuation of the valve element brought about by mechanical contact, according to the invention—in simple terms—a fluid-pressure-induced actuation is provided. By this means, the fluid passage can be shifted between the closed state and the open state depending on a fluid differential pressure prevailing in the fluid path between the inlet side and the outlet side. In contrast to customary catheter arrangements, release and sealing of the fluid-conducting path by means of the valve element is possible to this extent independently of a fluid-conducting component possibly connected proximally to the housing body. The fluid-conducting path can thereby be released and/or sealed solely depending on the prevailing fluid differential pressure independently of the presence or absence of such a fluid-conducting component. This firstly provides improved use safety and also permits a particularly simple design of the catheter arrangement. The fluid passage is made to extend through the elastic wall portion of the valve element. The fluid passage is preferably formed by at least one opening which is openable and closable in a manner induced by deformation and thus fluid pressure, in particular in the form of a slot and/or a slot arrangement with a plurality of slots. The elastic wall portion is elastically deformable differently depending on the sign and value of the prevailing fluid differential pressure. In the open state, the fluid-conducting path is released via the open fluid passage between the inlet side and the outlet side. In a state of the catheter applied on a patient, a medicinal fluid can thus be administered to the patient or blood taken from the patient, depending on the direction of flow through the fluid-conducting path. In the closed position, the fluid-conducting path is sealed fluid-tightly by means of the closed fluid passage. By means of the solution according to the invention, said fluid-tight seal, unlike in the case of conventional catheter arrangements with a valve actuation element, can also be present if a possible fluid-conducting component is connected to the proximal inlet side of the fluid-conducting path, more precisely: to the hollow housing body. In the state sealed fluid-tightly, in particular a blood return in the proximal direction is opposed. By this means, in particular an inadvertent escape of blood through the proximal inlet side is opposed. In order to ensure operation of the valve element so as to meet requirements, preferably at least the elastic wall portion is manufactured from an elastomeric material. Alternatively, the entire valve element can be manufactured from an elastomeric material. Suitable materials include in particular silicone, rubber or the like. The tube element may also be referred to as a catheter attachment. The hollow housing body may also be referred to as a catheter hub. The hollow needle may also be referred to as a canula. In accordance with customary medical parlance, the catheter arrangement may also be referred to as a peripheral indwelling venous catheter or peripheral indwelling venous canula. The tube element is preferably configured to be flexurally flexible. The tube element is preferably fitted directly onto the hollow housing body. For this purpose, a force-fitting, form-fitting and/or materially bonded joining connection formed between the housing body and the tube element Is preferably provided. For the force-fitting connection between the housing body and the tube element, a metal sleeve which is pressed proximally to the tube element can be provided. A suitable materially bonded joining connection can be in particular an adhesive connection. The tube element and/or the housing body are preferably manufactured from plastic. The housing body can be formed as a single part or in multiple parts. The housing body, at its proximal end facing away from the tube element, preferably has a connector portion for connection to a complementary connector portion of a medical fluid-conducting component, for example a medical syringe. The connector portion is preferably configured as a Luer connector. The hollow needle serves in a manner known to a person skilled in the art for venipuncture and for introducing the tube element into the punctured vein of the patient. When the catheter is applied in such a manner, the catheter arrangement takes up the state of readiness. After this, the hollow needle is drawn proximally out of the catheter, conventionally disposed of and the catheter arrangement is thereby transferred into the state of use. In the state of readiness, the hollow needle and the valve element preferably interact as follows: the hollow needle is made to extend through the fluid passage such that the latter is widened in the radial direction as a result of elastic deformation of the wall portion and bears fluid-tightly, in an elastically prestressed manner, against an outer circumference of the hollow needle. When the hollow needle is drawn out proximally, the elastic wall portion springs back inwards in the radial direction, as a result of which the fluid passage is closed.

In a refinement of the invention, the elastic wall portion is configured in such a manner that the fluid passage—in the state of use of the catheter arrangement and starting from its closed state—remains in the closed state in the event of a neutral fluid pressure, and can be shifted into the open state by means of an inlet-side fluid positive pressure and/or an inlet-side fluid negative pressure, wherein an inlet-side fluid negative pressure which is required for opening the fluid passage is greater in terms of value than an inlet-side fluid positive pressure which is required for the opening. Expressed in other words, the wall portion is elastically deformable to different extents depending on the direction of the pressurization. This results in a valve element behaviour that depends on the direction of the pressurization. In this refinement of the invention, the inlet-side fluid negative pressure which is required for opening the fluid passage is greater in terms of value than the inlet-side fluid positive pressure which is required for the opening. The inlet-side fluid negative pressure is equivalent to an outlet-side fluid positive pressure. This refinement of the invention ensures that flow can pass through the fluid-conducting path in the distal direction at comparatively lower fluid pressures than in the proximal direction. The elastic wall portion is preferably configured in such a manner that the inlet-side fluid positive pressure required for opening the fluid passage is achieved during a conventional gravity-driven infusion. By means of an appropriate configuration of the elastic wall portion, the inlet-side fluid negative pressure or outlet-side fluid positive pressure required for releasing the fluid-conducting path in the proximal direction is higher than the inlet-side fluid positive pressure which is required for releasing the fluid-conducting path in the distal direction. By this means, an inadvertent blood return in the proximal direction and in particular an escape of blood in the proximal direction is opposed in a simple and particularly effective manner. In order to achieve the previously described, direction-dependent behaviour, the elastic wall portion can be configured in particular in the shape of a small hat, a cup, a funnel, a dome, a cupola or a spherical cap.

In a further refinement of the invention, the inlet-side fluid negative pressure which is required for opening the fluid passage exceeds the inlet-side fluid positive pressure which is required for opening the fluid passage by 15 times to 25 times, preferably by 20 times. Expressed in other words, in this embodiment of the invention, the elastic wall portion is configured in such a manner that the fluid passage can be opened in the direction of the patient in the event of a comparatively small positive pressure. By contrast, the fluid passage can be opened in the event of a comparatively high inlet-side fluid negative pressure and thus equivalently in the case of comparatively high vein-side fluid positive pressures. This affords numerous advantages in the use of the catheter arrangement.

In a further refinement of the invention, the inlet-side fluid positive pressure which is required for opening the fluid passage is between 0.2 PSI and 0.4 PSI, preferably 0.3 PSI, and the inlet-side fluid negative pressure which is required for opening the fluid passage is between 5.0 PSI and 7.0 PSI, preferably 6.0 PSI. The previously mentioned value ranges have proven particularly advantageous in practice and are achieved by a corresponding configuration of the elastic wall portion. By means of the previously mentioned value ranges, in particular reliable opening of the fluid passage during a conventional gravity infusion is achieved. In addition, the fluid passage is prevented from inadvertently opening due to patient-side influences, for example a movement of the patient, sneezing, coughing, vomiting and the associated, physiologically increased outlet-side (vein-side) fluid positive pressure.

In a further refinement of the invention, the elastic wall portion has a cupola-shaped curvature, wherein the fluid passage is arranged in the region of an apex point of the curvature. A behaviour of the valve element that is dependent on the direction of the fluid pressurization can be achieved in a structurally simple manner by the cupola-shaped curvature and the arrangement of the fluid passage in the region of the apex point. The shape of the elastic wall portion arising in this refinement of the invention may be referred to in particular as a cupola or dome. Furthermore preferably, the apex point of the curvature is arranged on an imaginary central longitudinal axis of the fluid-conducting path.

In a further refinement of the invention, the cupola-shaped curvature of the elastic wall portion is concave in the direction of the proximal inlet side and convex in the direction of the distal outlet side. This orientation of the cupola-shaped curvature in particular ensures that the fluid-conducting path can be released comparatively “more easily” in the distal direction than in the proximal direction.

In a further refinement of the invention, the cupola-shaped curvature of the elastic wall portion is convex in the direction of the proximal inlet side and concave in the direction of the distal outlet side. The inventors have found that, by this means, a less turbulent or even laminar flow through the fluid passage in the direction of the proximal inlet side can be achieved. Expressed in other words, the curvature of the elastic wall portion which is convex in the proximal direction and concave in the distal direction permits a low-turbulence or even turbulence-free aspiration of liquid. If the catheter arrangement is used for taking blood, haemolysis can thereby be avoided in a simple, but particularly effective manner. Haemolysis is understood as meaning the dissolution of red blood cells as a consequence of a mechanical destruction of the cell membrane. Such a mechanical destruction can take place, for example, as a consequence of a turbulent flow and the forces occurring in the process. In addition, the present refinement permits a comparatively turbulent flow in the distal direction. By this means, for example in the case of an infusion directed in the distal direction, an accumulation of germs putting health at risk in the region of the valve element can be avoided.

In a further refinement of the invention, the valve element has a radially outer elastic articulated wall portion which is adjacent to the cupola-shaped curvature and, under the action of an infusion-induced fluid pressure and/or an aspiration-induced fluid pressure, permits an alternate sudden eversion of the cupola-shaped curvature between a stable first state, in which the cupola-shaped curvature is arched distally, and a stable second state, in which the cupola-shaped curvature is arched proximally. This refinement of the invention permits in particular a low-turbulence or even laminar flow through the fluid passage both in the distal and in the proximal direction. Advantages are thereby achieved both during an infusion and during an aspiration. In particular, the haemolysis already explained can be avoided. The articulated wall portion permits an alternate eversion, pushing out and/or buckling of the elastic wall portion together with the cupola-shaped curvature depending on the direction of the fluid flow. Depending on the direction of the fluid flow, the valve element “jumps” between the first state and the second state. Expressed in simplified terms, the valve element in this refinement has flip-flop properties. The articulated wall portion is preferably annular. Furthermore preferably, the cupola-shaped curvature is bounded in the circumferential direction by the, in particular annular, articulated wall portion. The articulated wall portion preferably has a smaller wall thickness in comparison to the cupola-shaped curvature.

In a further refinement of the invention, an axial height of the cupola-shaped curvature is smaller than a radial diameter of the elastic wall portion. Expressed in other words, the cupola-shaped curvature is comparatively flat. The inventors have found that advantageous flow properties can be achieved by this refinement of the invention. In particular, an accumulation of standing liquid at the outer edge of the convexly arched, cupola-shaped curvature is avoided. In association therewith, the accumulation of health-endangering germs and/or biofilms in said region is avoided.

As a result, this refinement of the invention permits improved patient safety. The axial height is preferably at maximum 50%, preferably at maximum 40%, particularly preferably at maximum 30%, of the radial diameter.

In a further refinement of the invention, the fluid passage is formed by a slot arrangement which has at least one first slot and a second slot, which slots are arranged preferably in a cross-shaped manner, particularly preferably in a “+”-shaped manner, forming at least one common intersecting point. The first slot and the second slot are oriented transversely, preferably perpendicularly, with respect to each other. The elastic wall portion is subdivided into different subsections by the slot arrangement. Said subsections are bounded and/or separated from one another by the first slot and/or the second slot. Depending on the prevailing fluid pressure, said subsections can be shifted relative to one another by elastic deformation. If the subsections are moved away from one another, the fluid passage is shifted into the open state. If the subsections lie fluid-tightly on one another, the fluid passage is in the closed state. This refinement of the invention is structurally simple and robust.

In a further refinement of the invention, the slot arrangement has a third slot, wherein the first slot, the second slot and the third slot are arranged in particular in a H-shaped manner, forming two common intersecting points. Such an, in particular H-shaped, arrangement permits in particular a comparatively large opening cross section of the fluid passage in the open state.

In a further refinement of the invention, the slot arrangement has a third slot, wherein the first slot, the second slot and the third slot are arranged in particular in a star-shaped manner, forming precisely one common intersecting point. The first slot, the second slot and the third slot are preferably in each case arranged offset by 120° with respect to one another such that a star-shaped configuration of the slot arrangement with a centrally arranged intersecting point is produced. The inventors have found that particular advantages can thereby be obtained. In a further refinement of the invention, the valve element has an encircling radial collar which is fixed in an encircling radial groove of the housing body, the housing body is configured as a single piece. The encircling radial collar can be manufactured separately from the elastic wall portion and then joined together therewith. Alternatively and preferably, the valve element is configured as a single piece such that the radial collar and the elastic wall portion are linked as a single piece. The radial groove is formed in the interior of the housing body and, for the installation of the valve element, is preferably accessible from the proximal inlet side. By means of the single-piece configuration of the housing body, a particularly simple design and, in association therewith, cost-effective manufacturing can be achieved.

In a further refinement of the invention, the elastic wall portion has at least one first pair of rib elements and one second pair of rib elements that are in each case arranged opposite one another in pairs with respect to the at least one common intersecting point, wherein the first pair of rib elements is arranged offset radially further outwards with respect to the intersecting point than the second pair of rib elements. The first pair of rib elements is preferably assigned to a first hollow needle with a first diameter. The second pair of rib elements is preferably assigned to a second hollow needle with a second diameter. The first diameter here is comparatively larger than the second diameter. The rib elements of the first pair may also be referred to as first rib elements. The rib elements of the second pair may also be referred to as second rib elements. Both the first and the second rib elements serve in each case for reinforcing regions of the elastic wall portion. The rib elements are preferably in each case linked as one piece with the elastic wall portion. In one refinement, the rib elements are manufactured as separate components and then fitted onto the elastic wall portion. The rib elements and the different spacing thereof from the intersecting point of the slot arrangement bring about an advantageous deformation behaviour of the elastic wall portion. The first rib elements and/or the first pair of rib elements bring about an advantageous deformation behaviour during use of the first hollow needle. The second rib elements and/or the second pair of rib elements bring about an advantageous deformation behaviour during use of the second hollow needle. The rib elements are preferably in each case arranged on a proximal side of the elastic wall portion. The rib elements preferably have a triangular shape, with in each case one point of the triangle being directed towards the intersecting point. In further refinements, the rib elements can have a shaping differing therefrom.

In a further refinement of the invention, at least one further fluid passage is made to extend through the elastic wall portion, wherein the further fluid passage is formed by at least two circumferential slots made to extend longitudinally in the circumferential direction of the elastic wall portion, and wherein the at least two circumferential slots are arranged offset radially outwards relative to the fluid passage. The inventors have found that this refinement of the invention permits an even better administration of liquid (infusion) and removal of liquid (aspiration) through the valve element. The further fluid passage formed by the at least two circumferential slots—basically corresponding to the fluid passage — can be transferred between an open and a closed state depending on the prevailing fluid pressure. The properties of the fluid passage and of the further fluid passage are preferably coordinated with one another in such a manner that a fluid-pressure-induced opening and closing takes place in an alternating manner. For example, the fluid passage can be closed during an aspiration and the further fluid passage opened. Conversely, during an infusion, the further fluid passage is preferably closed and the fluid passage opened. Of course, a refinement which is the other way around to this is also possible. The corresponding fluid-pressure-induced opening and closing properties of the fluid passage and of the further fluid passage are preferably achieved by an appropriate shaping, thickness and/or selection of material in the region of the fluid passage and the further fluid passage. The at least two circumferential slots are preferably arranged offset with respect to one another by 180° in the circumferential direction.

In a further refinement of the invention, the circumferential slots are in each case longer on a distal side of the elastic wall portion than on a proximal side of the elastic wall portion, and/or the fluid passage has at least one slot which is longer on the proximal side of the elastic wall portion than on the distal side of the elastic wall portion. Accordingly, the at least two circumferential slots are not designed approximately rectilinearly, but rather in an inclined manner with respect to one another on their end regions lying opposite one another in the longitudinal direction. This inclination brings about said difference in length of the circumferential slots between the distal and the proximal side. Said difference in length and/or inclination of the end regions causes the circumferential slots to be comparatively more easily releasable, that is to say at lower pressures, during an aspiration of liquid (in the proximal direction) than during an infusion (in the proximal direction). A corresponding statement applies analogously in respect of the at least one slot of the fluid passage. In a further refinement of the invention, the length ratios of the circumferential slots and of the at least one slot are designed the other way around to the preceding refinement.

In a further refinement of the invention, the elastic wall portion has at least one recessed or raised profiling in the region of the fluid passage, and/or the elastic wall portion has at least one recessed or raised further profiling in the region of the further fluid passage. The profiling and/or further profiling enables an improved directional dependency in the opening and closing behaviour f the fluid passage and/or further fluid passage to be achieved. If the profiling and/or further profiling is raised, a local increase in regions of the wall thickness of the elastic wall portion is thereby achieved. Consequently, the elastic wall portion is locally stiffened and/or reinforced. If the profiling and/or further profiling is recessed, this achieves a local reduction in regions of the wall thickness of the elastic wall portion. The wall thickness which is reduced in this manner brings about a local weakening.

In a further refinement of the invention, the valve element has a radial collar which is fixed between two joined-together housing parts of the housing body. By means of the at least two-part configuration of the housing body, a comparatively simple installation of the valve element can be achieved. For this purpose, the radial collar of the valve element is fixed in the axial direction between the two housing parts. The housing parts are then fixedly joined together in a manner known to a person skilled in the art, for example by means of an adhesively bonded or welded connection.

The invention also relates to a valve element for a catheter arrangement, having an elastic wall portion through which a fluid passage is made to extend, wherein the wall portion is elastically deformable under the action of a fluid pressure, and wherein the fluid passage can be shifted by means of a fluid-pressure-induced elastic deformation of the wall portion between an open state, in which the fluid passage is open, and a closed state, in which the fluid passage is closed. With regard to advantages associated with the configuration according to the invention of the valve element, what has been stated with regard to the catheter arrangement according to the invention is noted and express reference is made thereto. What has been disclosed to this extent with regard to the catheter arrangement according to the invention also applies mutatis mutandis to the valve element according to the invention. Refinements of the valve element according to the invention emerge from the features of the valve element of the refinements of the catheter arrangement according to the invention.

### BRIEF DESCRIPTION OF THE DRAWING FIGURES

Further advantages and features of the invention emerge from the claims and from the description below of preferred exemplary embodiments of the invention which are illustrated with reference to the drawings.

FIG. 1 shows, in a schematic perspective illustration, an embodiment of a catheter arrangement according to the invention with a catheter and a hollow needle, wherein the catheter arrangement is in a state of readiness.

FIG. 2 shows the catheter arrangement according to FIG. 1, wherein the hollow needle has been drawn out of the catheter in the proximal direction and is arranged separately therefrom.

FIG. 3 shows, in a schematically highly simplified and partially cut-away longitudinal sectional illustration, a hollow housing body of the catheter with a valve element arranged therein.

FIG. 4 shows, in an illustration corresponding to FIG. 3, an alternative refinement of the hollow housing body.

FIG. 5 shows an enlarged illustration of a detail of the valve element in a sectional view corresponding to FIGS. 3 and 4.

FIG. 6 shows the valve element according to FIG. 5 in a direction looking axially at a fluid passage.

FIG. 7 shows, in an illustration corresponding to FIG. 6, an embodiment of a valve element according to the invention with an alternatively configured fluid passage.

FIG. 8 shows, in an illustration corresponding to FIGS. 6 and 7, a further embodiment of a valve element according to the invention with an alternatively configured fluid passage.

FIG. 9 shows, in a schematic longitudinal sectional illustration, a further embodiment of a valve element according to the invention with a cupola shape of flat design.

FIG. 10 shows, in a schematic perspective illustration, a further embodiment of a valve element according to the invention with a further fluid passage formed from two circumferential slots.

FIG. 11 shows a greatly simplified schematic sectional illustration for clarifying further features of the valve element according to FIG. 10.

FIG. 12 shows, in a perspective sectional illustration, a further embodiment of a valve element according to the invention.

FIG. 13 shows, in a perspective sectional illustration, a further embodiment of a valve element according to the invention,

FIG. 14 shows, in a schematic perspective illustration, a further embodiment of a valve element according to the invention which can be transferred in a fluid-pressure-induced manner between a stable first state (FIG. 16) and a stable second state (FIG. 17).

FIG. 15 shows, in a schematic longitudinal sectional illustration, the valve element according to FIG. 14.

FIGS. 16 and 17 show the valve element according to FIGS. 14 and 15 in said first state (FIG. 16) and the second state (FIG. 17).

FIG. 18 shows, in a schematic perspective illustration, a further embodiment of a valve element according to the invention.

FIG. 19 shows the valve element according to FIG. 18 in a further perspective view with a direction looking at a proximal wall side of the elastic wall portion.

FIG. 20 shows a further embodiment in a schematic side view.

### DETAILED DESCRIPTION

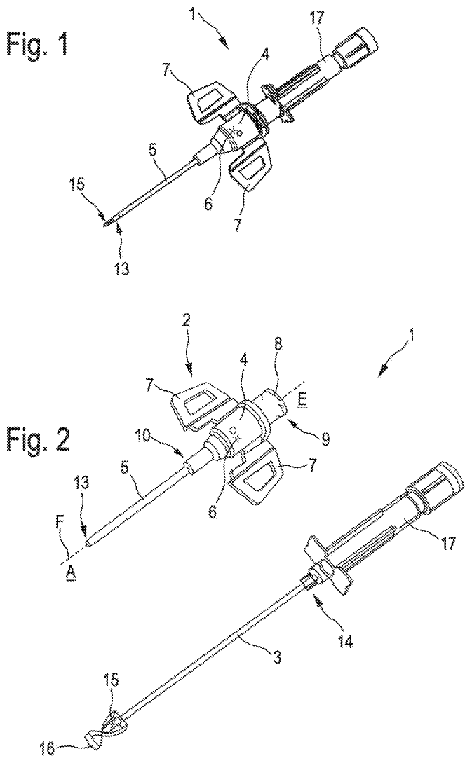

According to FIGS. 1 and 2, a catheter arrangement 1 for use in infusion therapy is provided and has a catheter 2 and a hollow needle 3. The catheter arrangement 1 may also be referred to as peripheral indwelling venous canula or peripheral indwelling venous catheter. The catheter arrangement 1 is applied in a manner known to a person skilled in the art in the region of the back of a hand or a crook of the arm of a patient and serves in particular for parenteral liquid therapy, an intravenous administration of medicaments, and/or for taking blood.

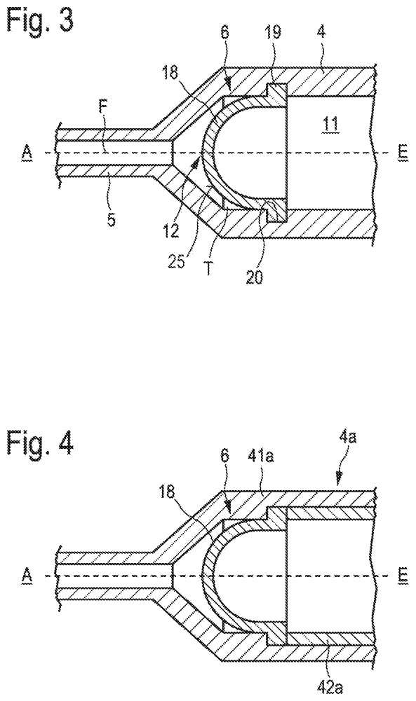

The catheter 2 has a hollow housing body 4, a tube element 5 and a valve element 6 (FIG. 3). In FIG. 3, the housing body 4 and the tube element 5 are provided with continuous hatching. This does not necessarily mean that the housing body 4 and the tube element 5 are linked as a single piece.

The housing body 4 may also be referred to as a catheter hub and has a basically known basic shape with two laterally protruding fastening wings 7 and a connector portion 8.

In an embodiment which is not illustrated graphically, the housing body does not have fastening wings.

The connector portion 8 is arranged at a proximal end 9 of the housing body 4 and is configured in the present case in the form of a female Luer lock connection. The tube element 5 is arranged at a distal end 10 of the housing body 4 and is joined together fixedly to the housing body 4 in a manner known to a person skilled in the art. For example, the tube element 5 can be joined together for this purpose to the housing body 4 by means of a press connection, welded connection or adhesively bonded connection. A metal sleeve can be provided for the press connection and, by expansion of the tube element 5, can be pressed into the proximal end thereof. In addition, a configuration of the housing body linked in a single piece with the tube element is possible.

The catheter 2 has a fluid-conducting path F (FIGS. 2 and 3) which is made to extend through the catheter 2 in an axial direction of the catheter 2 between a proximal inlet side E and a distal outlet side A. The fluid-conducting path F runs here from the distal end 9 into the housing body 4, more specifically: into a cavity 11 of the housing body 4, from there further in the distal direction through a fluid passage 12 of the valve element 6, and from there through the tube element 5 and the point 13 thereof as far as the outlet side A.

The fluid passage 12 can be shifted between a closed state and an open state in a manner described in yet more detail. In the closed state, the fluid passage 12 is closed and the fluid-conducting path F between the inlet side E and the outlet side A is thereby sealed fluid-tightly. In the open state, the fluid passage 12 is opened and the fluid-conducting path F between the inlet side E and the outlet side A is thereby released.

The hollow needle 3 is made to extend longitudinally between a proximal end 14 and a distally arranged needle point 15 which, in the state apparent with reference to FIG. 2, is covered by means of a safety element 16 in a manner known to a person skilled in the art. The hollow needle 3 is joined together at its proximal end to a needle attachment 17. The needle attachment 17, like the hollow needle 3, is configured in a manner known to a person skilled in the art. To this extent, in particular a further explanation of the configuration here of the needle attachment 17 can be omitted.

The state shown with reference to FIG. 1 illustrates a state of readiness of the catheter arrangement 1, in which the hollow needle 3 is plugged from the inlet side E in the distal direction into the catheter 2. The hollow needle 3 is made to extend here through the housing body 4, the fluid passage 12 of the valve element 6 and the tube element 5, with the needle point 15 protruding over the point 13 in the distal direction.

In order to apply the catheter 2, the catheter arrangement 1 in its state of readiness is brought up to an appropriate vein of the patient and the vein is punctured by means of the needle point 15. The hollow needle 3 is pushed together with the tube element 5 into the punctured vein. The hollow needle 3 is then drawn out of the catheter 2 in the proximal direction, conventionally disposed of and the catheter arrangement 1 is thereby transferred into a state of use. In said state of use, the catheter 2 is applied to the patient and generally usable for several days. In the state of use, the fluid-conducting path F is either sealed fluid-tightly or released by means of the sealing element 6, depending on the use situation of the catheter 2.

The valve element 6 has an elastic wall portion 18. The fluid passage 12 is made to extend in a form described in yet more detail through the elastic wall portion 18 in the thickness direction thereof. The elastic wall portion 18 is elastically deformable under the action of a fluid pressure acting thereon. As a result of said fluid-pressure-induced elastic deformation, the fluid passage 12—at any rate in the state of use of the catheter arrangement 1—can be shifted between its open state and its closed state. In the open state, the fluid passage 12 is open such that the fluid-conducting path F between the inlet side E and the outlet side A is released. In the closed state, the fluid passage 12 is closed, as a result of which the fluid-conducting path F is sealed fluid-tightly by means of the closed fluid passage 12. In contrast to solutions known from the prior art, the valve element 6 can thus be opened and closed solely owing to the fluid pressure conditions prevailing in the fluid-conducting path F.

In the embodiment shown, the elastic wall portion 18 is configured in such a manner that different fluid pressures for opening the fluid passage 12 are required depending on the direction of passage through the fluid-conducting path F. For further explanation, it is assumed that, on the inlet side, a fluid pressure p_{E} and, on the outlet side, a fluid pressure p_{A} prevail in the fluid-conducting path F. The inlet-side fluid pressure p_{E} acts on a proximal wall side 24 of the elastic wall portion 18, said proximal wall side 24 facing the inlet side E. The outlet-side fluid pressure p_{A} acts on a distal wall side 25 of the elastic wall portion 18, said distal wall side 25 facing the outlet side A. Starting from its closed state, the fluid passage 12 remains closed in the event of a neutral fluid pressure, i.e. p_{E}=p_{A}. If the inlet-side fluid pressure p_{E} exceeds the outlet-side fluid pressure p_{A}, an inlet-side fluid positive pressure Δp_{E} is present. An inlet-side fluid positive pressure Δp₁ is required for opening the fluid passage 12. This pressure may also be referred to as the required inlet-side fluid positive pressure or inlet-side opening positive pressure Δp₁.

If the inlet-side fluid pressure p_{E} falls short of the outlet-side fluid pressure p_{A}, an inlet-side fluid negative pressure −Δp_{E} is present. This is equivalent to an outlet-side fluid positive pressure Δp_{A}. An inlet-side fluid negative pressure −Δp₂ is required for opening the fluid passage 12. With regard to the outlet side A, it can also be mentioned that an outlet-side fluid positive pressure Δp₃ is required for opening the fluid passage 12. This may also be referred to as the outlet-side opening positive pressure Δp₃.

The fluid differential pressures Δp₁, −Δp₂ and Δp₃ that are required for opening the fluid passage 12 differ depending on the direction of fluid pressurization of the elastic wall portion 18. In the embodiment shown, the required outlet-side fluid positive pressure Δp₃ is greater than the required inlet-side fluid positive pressure Δp₁. Expressed in other words, the required inlet-side fluid negative pressure −Δp₂ is greater in terms of value than the required inlet-side fluid positive pressure Δp₁.

Furthermore, it is understood that, in the clinical use of the catheter arrangement, states with a neutral fluid pressure, i.e. p_{E}=p_{A}, cannot occur. This is because of the vein pressure which always exists and which acts on the outlet side on the elastic wall portion 18.

In the embodiment shown, the elastic wall portion 18 is configured in such a manner that the inlet-side opening positive pressure Δp₁ is 0.3 PSI. By this means, in particular opening of the fluid passage in a manner meeting requirements is achieved during conventional gravity infusion. In addition, the elastic wall portion 18 here is configured in such a manner that the inlet-side opening negative pressure Δp₂is 6 PSI. Such a negative pressure can be readily applied by means of a medical syringe connected to the connector portion 8 such that a blood aspiration meeting requirements is ensured. At the same time, inadvertent opening of the fluid passage 12 as a result of physiological phenomena on the part of the patient is opposed.

The previously described direction-dependent opening and closing behaviour of the fluid passage 12 is achieved by a configuration, which is described in yet more detail, of the elastic wall portion 18 and of the fluid passage 12 arranged therein.

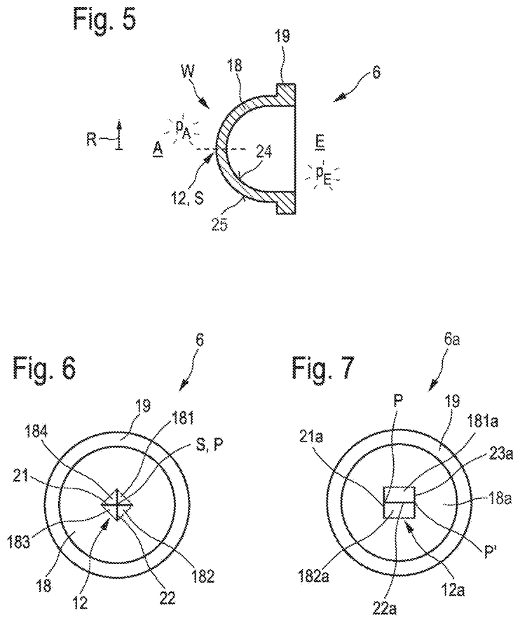

For this purpose, it is provided here that the elastic wall portion 18 has a cupola-shaped curvature W (FIG. 5). The fluid passage 12 is arranged in the region of an apex point S of the curvature W. The cupola-shaped curvature W is curved outwards in the direction of the outlet side A and inwards in the direction of the inlet side E. Expressed in other words, the cupola-shaped curvature W is configured to be concave in the direction of the proximal inlet side E and convex in the direction of the distal outlet side A. In the embodiment shown, the elastic wall portion 18 has a constant wall thickness, and therefore the wall sides of the elastic wall portion 18 that are opposite one another in the thickness direction are made to extend in parallel.

In further embodiments, the elastic wall portion does not have a constant wall thickness.

As a result of the cupola-shaped curvature W, the valve element 6 has a configuration which, in visual language, may also be referred to as cupola-shaped, dome-shaped or approximately hemispherical. The valve element E here is rotationally symmetrical and is so with respect to a line of symmetry which coincides with the fluid-conducting path F that is shown schematically in FIG. 3.

On its outer circumference, the valve element 6 has a radial collar 19 which encircles in the circumferential direction and protrudes from the elastic wall portion 18 in the radial direction R. The radial collar 19 is fixed in a radial groove 20 of the hollow housing body 4 (FIG. 3). The housing body 4 is configured as a single piece here.

In the embodiment shown, the entire valve element 6 is manufactured from an elastomeric material, for example silicone. This is not absolutely necessary. In an embodiment which is not illustrated graphically, only the elastic wall portion 18 is manufactured from an elastomeric material.

For the installation, the valve element 6 is introduced from the inlet side E in the distal direction into the cavity 11. In the process, the valve element 6 is slightly elastically compressed in the radial direction R. As soon as the radial collar 19 enters the region of the radial groove 20, the valve element 6 springs outwards in the radial direction R. By this means, the radial collar 19 enters into form-fitting engagement with the radial groove 20, as a result of which the valve element 6 is fixed captively in the housing body 4.

In the alternative embodiment shown with reference to FIG. 4, the housing body 4a has a two-part configuration with a first housing part 41a and a second housing part 42a. The valve element 6 is fixed between the two housing parts 41a, 42a. For the installation, the valve element 6 is pushed in the axial direction from the inlet side E into the first housing part 41a. An elastic deformation of the valve element 6 is not necessarily required here. After the valve element 6 is introduced, the second housing part 42a is pushed axially and in the distal direction into the first housing part 41a and the valve element 6 is thereby fixed between mutually axially opposite end surfaces of the first housing part 41a and of the second housing part 42a. The two housing parts 41a, 42a are then joined together in a manner known to a person skilled in the art, for example are adhesively bonded or welded.

Instead 42a being a catheter housing, it can be a representative of a tube valve (for example, a silicone valve found in a Ported IVC) or integrated IVC.

In particular, the fluid passage may differ in configuration. In the simplest case, the fluid passage is formed by an individual slot.

In the configuration according to FIG. 6, the fluid passage 12 is formed by a slot arrangement 21, 22 which has a first slot 21 and a second slot 22. The first slot 21 and the second slot 22 are each made to extend as radial slots in the radial direction R and form a common intersecting point P. In the embodiment shown, the latter coincides with the apex point S of the cupola-shaped curvature W. The first slot 21 and the second slot 22 are arranged in a cross-shaped manner with respect to each other and are oriented with respect to each other here. The slot arrangement 21, 22 thereby assumes a “+”-shaped configuration. The slot arrangement 21, 22 is made to extend in the axial direction through the wall thickness of the elastic wall portion 18. The fluid passage 12 is shown here with reference to FIG. 6 in its closed state. In said closed state, subsections 181, 182, 183, 184 of the elastic wall portion 18, which subsections 181, 182, 183, 184 are separated from one another by the slot arrangement 21, 22, lie fluid-tightly against one another. The subsections 181 to 184 are in each case approximately triangular, which is intended to be clarified schematically by the dashed lines shown in FIG. 6. During a corresponding fluid pressurization of the elastic wall portion 18, the subsections 181 to 184 are arched and unfolded in the axial direction relative to the remaining portions of the elastic wall portion, as a result of which the fluid passage 12 is opened. This arching or unfolding of the subsections 181 to 184 takes place either in the direction of the outlet side A or in the direction of the inlet side E depending on the prevailing fluid pressure conditions.

Further embodiments of valve elements 6a to 6i according to the invention are shown with reference to FIGS. 7 to 20. The valve elements 6a to 6i are substantially identical in respect of their design and their operation to the valve element 6. In order to avoid repetitions, primarily substantial differences of the valve elements 6a to 6i will therefore be explained below. Otherwise, what has been already disclosed with regard to the valve element 6 is noted and express reference is made thereto. The valve elements 6a to 6i can be used instead of the valve element 6 for the catheter arrangement 1.

The valve element 6a according to FIG. 7 provides a slot arrangement 21a, 22a, 23a with a first slot 21a, a second slot 22a and a third slot 23a. Said slots are arranged so as to form two common intersecting points P, P′ and, in the embodiment shown, form a H shape. The slot arrangement 21a, 22a, 23a separates subsections 181a, 182a of the elastic wall portion 18a. Said subsections have an approximately rectangular configuration, which is clarified in turn by the dashed lines. The fluid passage 12a formed by the slot arrangement 21a, 22a, 23a is shown in its closed state in FIG. 7. Upon shifting into the open state, the subsections 181a, 182a unfold elastically in the axial direction.

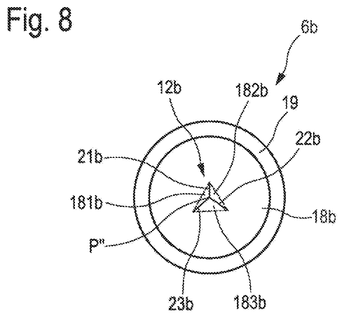

The valve element 6b shown with reference to FIG. 8 provides a slot arrangement 21b, 22b, 23b with a first slot 21b, a second slot 22b and a third slot 23b. These slots are arranged so as to form precisely one common intersecting point P″ and, in the embodiment shown, form a star shape. The slot arrangement 21b, 22b, 23b separates subsections 181b, 182b, 183b of the elastic wall portion 18b. Said subsections have an approximately triangular configuration, which is clarified in turn by the dashed lines. The fluid passage 12b formed by the slot arrangement 21b, 22b, 23b is shown in its closed state in FIG. 8. Upon shifting into the open state, said subsections 181b, 182b, 183b unfold elastically in the axial direction.

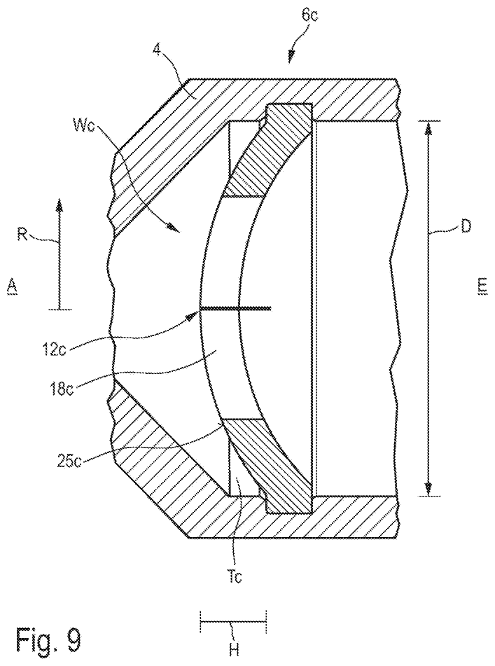

In contrast to the valve element 6, the valve element 6c according to FIG. 9 has a comparatively flat cupola-shaped curvature Wc. This is achieved by a maximum height H of the cupola-shaped curvature Wc being smaller than a radial diameter D of the elastic wall portion 18c. In the embodiment shown, a ratio between the axial height H and the radial diameter D is approximately 1:5. The valve element 6c has improved flow properties in comparison to the valve element 6. To this end, reference is made to a direct comparison between FIGS. 3 and 9. In the fitting situation shown with reference to FIG. 3, what is referred to as a dead space is identified by reference sign T. The dead space T extends annularly on a radially outer region of the distal wall side 25 of the valve element 6. Even in the open state of the valve element 6 an only slow or, in the worst case, even no fluid flow arises in the dead space T. The dead space T is relatively narrow because of the comparatively pronounced cupola-shaped curvature W and therefore the flow only passes weakly or, in the worst case, does not pass therethrough. For this purpose, by contrast, the fitting situation of the valve element 6c shown with reference to FIG. 9 has a dead space Tc which is relatively large and thus the flow can pass therethrough comparatively readily. This is because of the comparatively flat cupola-shaped curvature Wc. The flat cupola-shaped curvature Wc permits an improved fluid flow in the radially outer region of the distal wall side 25c. In the simplest case, the fluid passage 12c can be formed by a single longitudinal slot. Alternatively, a configuration of the fluid passage 12c as a slot arrangement according to the preceding and/or following description is possible.

In a further embodiment, dead space T may also be reduced by shaping the catheter hub to conform to the shape of the valve. For example, the catheter hub may have a curved profile conforming with the dome or cupola shape of the valve. This helps to minimise dead space T and enhance the alignment of the valve and catheter hub during the assembly process. To minimise the dead space, the valve may be shaped to conform to the shape of the inner profile of the catheter hub. The catheter hub may also be shaped to conform to the shape of the valve.

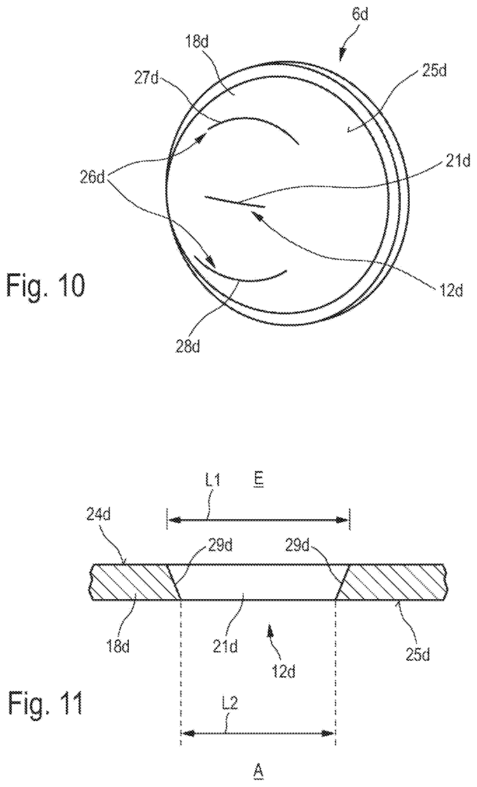

In contrast to the previously described embodiments, the valve element 6d according to FIG. 10 has a further fluid passage 26d. The further fluid passage 26d is made to extend through the elastic wall portion 18d and can be transferred in a manner induced by fluid pressure between an open state and a closed state. This basically corresponds to the fluid passage 12d which, in the embodiment shown, is formed by a single first slot 21d. The further fluid passage 26d has at least two circumferential slots 27d, 28d, which may also be referred to as the first circumferential slot 27d and second circumferential slot 28d. The circumferential slots 27d, 28d are made to extend in the circumferential direction of the elastic wall portion 18d which here is curved in a cupola-shaped manner. The circumferential slots 27d, 28d are arranged offset by 180° with respect to one another in the circumferential direction. The circumferential slots 27d, 28d are arranged mirror-symmetrically opposite one another and/or with respect to the fluid passage 12d, more precisely: the slot 21d thereof. The circumferential slots 27d, 28d are arranged offset outwards in the radial direction relative to the fluid passage 12d. The first slot 21d is in turn arranged in an apex point, not denoted specifically, of the cupola-shaped curvature.

In a further embodiment, instead of being oriented in the circumferential direction, the slots may be straight cuts and may be arranged in a radially manner.

It is shown with reference to FIG. 11 that the slot 21d of the fluid passage 12d has a proximal slot length L1 on the proximal wall side 24d. On the distal wall side 25d, the slot 21d has a different distal slot length L2. In the embodiment shown, the proximal slot length L1 is greater than the distal slot length L2. This assists an infusion-pressure-induced opening of the fluid passage 12d. For this purpose, by contrast, a converse ratio between the proximal slot length L1 and the distal slot length L2 would assist an aspiration-pressure-induced opening of the fluid passage 12d. Owing to the different slot lengths L1, L2, end regions which are opposite one another in the longitudinal direction of the slot 21d are inclined with respect to one another.

The circumferential slots 27d, 28d of the further fluid passage 26d have slot length ratios which are the other way around compared to the slot 21d. Expressed in other words, the circumferential slots 27d, 28d are in each case longer on the distal wall side 25d than on the proximal wall side 24d. This simplifies aspiration of liquid through the further fluid passage 26d.

In one use of the valve element 6d, the operation thereof is in particular as follows. During an infusion of liquid (fluid flow in the distal direction) and an infusion pressure prevailing in this connection, the fluid passage 12d is shifted into its open state. In this case, the further fluid passage 26d preferably remains in its closed state. When the infusion pressure is increased, the further fluid passage 26d can be additionally shifted into its open state. During an aspiration of liquid (fluid flow in the proximal direction) and an aspiration pressure prevailing in this connection, the further fluid passage 26d is shifted into its open state. The fluid passage 21d remains here in its closed state. The previously described direction-dependent opening and closing behaviour of the fluid passage 12d and of the further fluid passage 26d is assisted by said slot length ratios.

Furthermore, it is understood that the fluid passage 12d can alternatively be formed by a slot arrangement according to the preceding or following description. In addition, the further fluid passage 26d can be formed by fewer or more than the two circumferential slots 27d, 28d shown here. For example, three, four, five, six or more than six circumferential slots are conceivable.

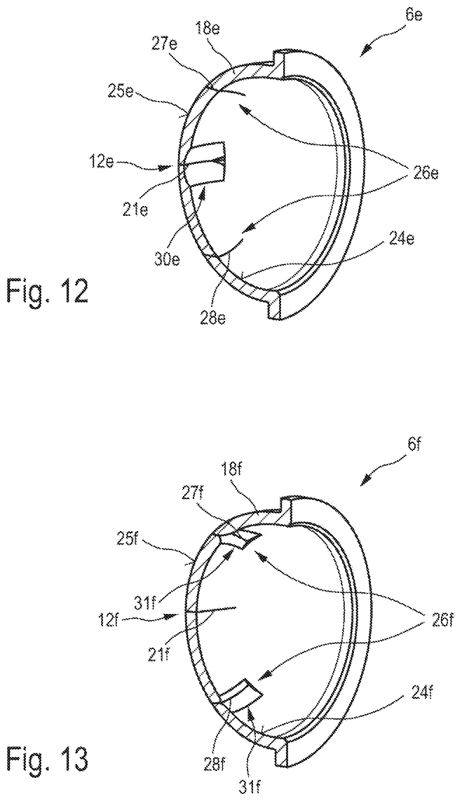

The valve element 6e according to FIG. 12 differs by means of a profiling 30e which is present in the region of the fluid passage 12e. The profiling 30e is formed on the elastic wall portion 18e. The profiling 30e can basically be designed as a raised or recessed profiling. In the present case, the profiling 30e is recessed in the elastic wall portion 18e. The profiling 30e is recessed in the elastic wall portion 18e from the proximal wall side 24e. If the profiling instead is designed as a raised profiling, the profiling preferably protrudes from the elastic wall portion 18e starting from the distal wall side 25e. The profiling 30e leads to a reduced wall thickness of the elastic wall portion 18e in the region of the fluid passage 12e. The profiling 30e may also be referred to as an indentation, hollow, groove, channel or the like. The profiling 30e assists an infusion-pressure-induced opening of the fluid passage 21e.

Furthermore, the fluid passage 12e has only one single first slot 21e. It is understood that the fluid passage 12e in further refinements can be formed by a slot arrangement according to the preceding or following description. In this case, the profiling is adapted to the specific refinement of the fluid passage.

In addition, the valve element 6e in accordance with the valve element 6d according to FIGS. 10 and 11 has a further fluid passage 26e, but this is not compulsory.

The valve element 6f according to FIG. 13 differs from the valve element 6e according to FIG. 12 in that further profilings 31f are present in the region of the further fluid passage 26f. The further profilings 31f are in each case arranged on the first circumferential slot 27f and the second circumferential slot 28f and influence the opening and closing behaviour of said circumferential slots. The further profilings 31f —corresponding to the profiling 30e—can be basically raised or recessed. In addition, the further profilings 31f can be arranged on the proximal wall side 24f and/or the distal wall side 25f of the elastic wall portion 18f. In the embodiment shown, the further profilings 31f are arranged on the proximal wall side 24f and are recessed in same. In a further refinement, the two further profilings are arranged and formed in a raised manner on the distal wall side 25f. In a further refinement, one of the further profilings is arranged on the proximal wall side and a further of the further profilings on the distal wall side.

It is understood that the profilings 30e, 31f shown with reference to FIGS. 12 and 13 for influencing the opening and closing behaviour of the fluid passage and of the further fluid passage can be combined and formed differently with one another. In addition, a combination with the different slot lengths shown with reference to FIG. 11 on the proximal and distal wall side is possible.

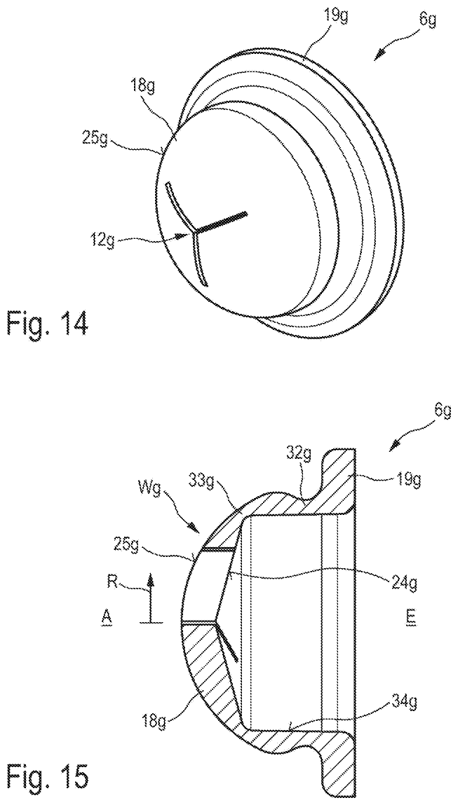

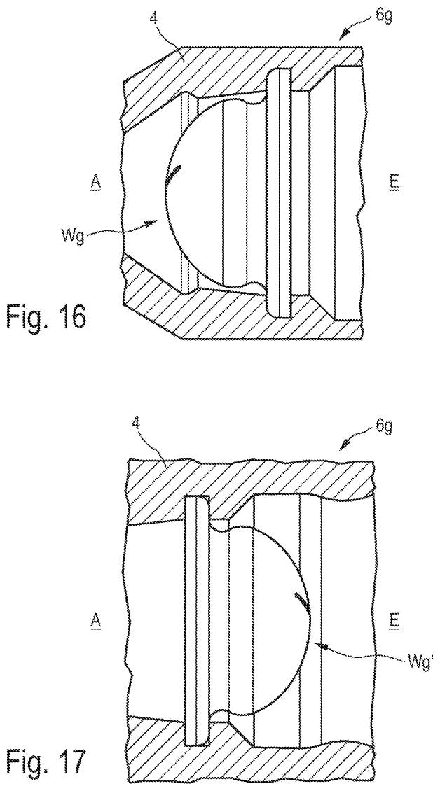

The valve element 6g (FIGS. 14, 15) permits an alternate, sudden eversion of the elastic wall portion 18g between a first state (FIG. 16) and a second state (FIG. 17). The sudden eversion takes place depending on the respective pressurization and/or direction of the fluid flow. Expressed in simplified form, the valve element 6g has flip-flop characteristics—it flips and flops based on fluid direction.

For this purpose, the valve element 6g has an elastic articulated wall portion 32g. The elastic articulated wall portion 32g is arranged lying radially on the outside and at one end borders the elastic wall portion 18g and at the other end the radial collar 19g. The elastic articulated wall portion 32g is of annular design. In the region of the elastic articulated wall portion 32g the valve element 6g has a reduced wall thickness—in comparison to the adjacent wall portions. This reduced wall thickness permits said eversion and/or switching-over movement of the valve element 6g.

It can furthermore be seen with respect to FIG. 15 that the elastic wall portion 18g has a wall thickness which increases in the radial direction R from the outside inwards and which is at its maximum approximately in the region of the apex point of the cupola-shaped curvature Wg. The wall thickness increasing from the outside inwards permits an improved stability of the elastic wall portion 18g both in the first state (FIG. 16) and in the second state (FIG. 17). The radially outer region with a minimal wall thickness may also be referred to as the annular wall portion 33g. The annular wall portion 33g assists an eversion meeting requirements because of its comparatively low wall thickness. Furthermore, the valve element 6g has a substantially cylindrical cylinder wall portion 34g (FIG. 15). The cylinder wall portion 34g, at any rate in the first state, is arranged on a radially inner side of the valve element 6g and at one end borders the proximal wall side 24g.

In the first state, the cupola-shaped curvature Wg is arched distally. Expressed in other words, the elastic wall portion 18g is concave in the first state in the direction of the proximal inlet side E and convex in the direction of the distal outlet side A.

In the second state, the elastic wall portion 18g is convex in the direction of the proximal inlet side and concave in the direction of the distal outlet side A. Accordingly, a cupola-shaped curvature Wg' which is inverted with respect to the cupola-shaped curvature Wg is present (FIG. 17).

In the use of the valve element 6g, the operation thereof is in particular as follows: starting from the first state (FIG. 16), an infusion pressure is applied in order to infuse liquid. The infusion pressure brings about an opening of the fluid passage 12g. The valve element 6g remains here in its first state. The first state is to this extent stable. By means of a reduction in the infusion pressure, the fluid passage 12g is shifted into its closed state. In order to aspirate liquid, an aspiration pressure is applied. This aspiration pressure brings about a sudden eversion of the elastic wall portion 18g into the second state (FIG. 17), wherein the fluid passage 12g initially remains closed here. If the aspiration pressure is maintained and/or increased, the fluid passage 12g is in turn shifted into its open state such that liquid can flow from the outlet side A to the inlet side E. The second state taken up here is again stable. By means of a reduction in the aspiration pressure, the fluid passage 12g is shifted into its closed state. By renewed application of an infusion pressure, the valve element 6g can again be everted into its first state and the fluid passage 12g opened again for the infusion of liquid.

The valve element 6g permits particularly advantageous flow properties. Both the infusion and the aspiration of liquid can take place with reduced turbulence or at best even completely laminarly. This is because of the previously described alternate sudden eversion between the first and second state. If the valve element 6g is used within the scope of taking blood, in particular a disadvantageous haemolysis can be avoided.

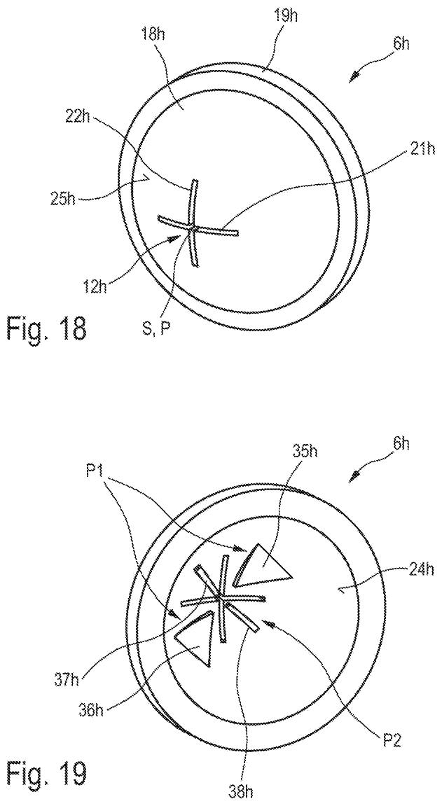

The valve element 6h according to FIGS. 18 and 19 differs from the valve elements shown up to now by a first pair P1 of rib elements and a second pair P2 of rib elements. The first pair of rib elements P1 has two rib elements 35h, 36h, which may also be referred to as the first rib element 35h and second rib element 36h. The second pair P2 of rib elements has two rib elements 37h, 38h, which may also be referred to as the third rib element 37h and fourth rib element 38h. The first rib element 35h and the second rib element 36h are arranged opposite each other in pairs with respect to the intersecting point P of the fluid passage 12h. The same applies analogously to the third rib element 37h and the fourth rib element 38h. The first pair P1 of rib elements is arranged offset radially further outwards with respect to the intersecting point P than the second pair P2 of rib elements.

The inventors have found that the hollow needle 3, which, in the state of readiness, is made to extend through the fluid passage 12h, may lead to an undesirable memory effect and thus to an at any rate partially remaining deformation of the elastic wall portion 18h. This memory effect is basically undesirable. The second pair P2 of rib elements opposes said memory effect. For this purpose, the rib elements 37h, 38h are arranged comparatively tightly against the intersecting point P of the fluid passage 12h. This results in a mechanical reinforcement of the elastic wall portion 18h which opposes said memory effect.

The first pair P1 arranged offset radially further outwards assists an elastic deformation, meeting requirements, of the elastic wall portion 18a during the aspiration and infusion of liquid.

In the embodiment shown, the first pair P1 and the second pair P2 and thus also all of the rib elements 35h, 36h, 37h, 38h are arranged on the proximal wall side 24h. In further refinements, at least one of the rib elements can be arranged on the distal wall side 25h. In addition, refinements are conceivable in which only one of the two pairs P1, P2 is present. Furthermore, the shaping of the rib elements that is shown with reference to FIG. 19 should be understood as being purely by way of example. In further refinements, a shaping different thereto can be provided.

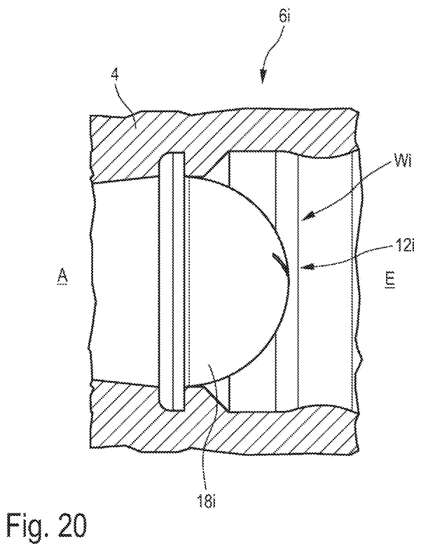

With reference to FIG. 20, a further embodiment is shown which—expressed in simplified terms—provides an inverted fitting of the valve element 6i into the housing body 4. The valve element 6i has in turn an elastic wall portion 18i with a cupola-shaped curvature Wi. The cupola-shaped curvature Wi is arched in the direction of the proximal inlet side E. Expressed in other words, the elastic wall portion 18i is convex in the direction of the proximal inlet side E and concave in the direction of the distal outlet side A. The inventors have found that the present embodiment permits improved flow properties during an aspiration of liquid. This is because, owing to the previously described orientation of the cupola-shaped curvature Wᵢ, reduced turbulences occur in the proximal flow direction. At best, even a laminar flow can be achieved. This opposes an undesirable haemolysis if the catheter arrangement is used for taking blood. Conversely, a turbulent flow in the distal direction is promoted. This prevents accumulations of germs.

It is obvious with reference to the preceding description that individual features of the valve elements 6 to 6i are considered in isolated form and can be combined with one another to form different combinations of features. For example, the flat cupola shape of the valve element 6c can be combined with the further fluid passage of the valve element 6d, the profilings of the valve elements 6e, 6f, the flip-flop properties of the valve element 6g and/or the rib elements of the valve element 6h.

1. A catheter arrangement, comprising:
a catheter comprising a hollow housing body, a tube element fitted onto a distal end of the housing body, a valve element which is arranged in the housing body and is provided with a fluid passage, and a fluid-conducting path which is made to extend longitudinally through the housing body, the fluid passage and the tube element between a proximal inlet side and a distal outlet side; and
a hollow needle which, in a state of readiness of the catheter arrangement, is made to extend longitudinally through the fluid-conducting path, and which, in a state of use of the catheter arrangement, is drawn out of the fluid-conducting path in the proximal direction,
the valve element has comprising an elastic wall portion through which the fluid passage is made to extend, and in that the wall portion is elastically deformable under the action of a fluid pressure, wherein the fluid passage—in the state of use of the catheter arrangement—is configured to be shifted by a fluid-pressure-induced elastic deformation of the wall portion between an open state, in which the fluid passage is open, and a closed state, in which the fluid passage is closed.

2. The catheter arrangement according to claim 1, wherein the elastic wall portion is configured in such a manner that the fluid passage—in the state of use of the catheter arrangement and starting from its closed state—remains in the closed state in the event of a neutral fluid pressure, and can be shifted into the open state by means of an inlet-side fluid positive pressure and/or an inlet-side fluid negative pressure, wherein an inlet-side fluid negative pressure which is required for opening the fluid passage is greater in terms of value than an inlet-side fluid positive pressure which is required for the opening.

3. The catheter arrangement according to claim 2, wherein the inlet-side fluid negative pressure which is required for opening the fluid passage exceeds the inlet-side fluid positive pressure which is required for opening the fluid passage by 15 times to 25 times.

4. The catheter arrangement according to claim 2, wherein the inlet-side fluid positive pressure which is required for opening the fluid passage is between 0.2PSI and 0.4 PSI and wherein the inlet-side fluid negative pressure which is required for opening the fluid passage is between 5.0 PSI and 7.0 PSI.

5. The catheter arrangement according to claim 1, wherein the elastic wall portion has a cupola-shaped curvature, and wherein the fluid passage is arranged in the region of an apex point of the curvature.

6. The catheter arrangement according to claim 5, wherein the cupola-shaped curvature of the elastic wall portion is concave in the direction of the proximal inlet side and convex in the direction of the distal outlet side.

7. The catheter arrangement according to claim 5, wherein the cupola-shaped curvature of the elastic wall portion is convex in the direction of the proximal inlet side and concave in the direction of the distal outlet side.

8. The catheter arrangement according to claim 5, wherein the valve element has a radially outer elastic articulated wall portion which is adjacent to the cupola-shaped curvature and, under the action of an infusion-induced fluid pressure and/or an aspiration-induced fluid pressure, permits an alternate sudden eversion of the cupola-shaped curvature between a stable first state, in which the cupola-shaped curvature is arched distally, and a stable second state, in which the cupola-shaped curvature is arched proximally.

9. The catheter arrangement according to claim 5, wherein an axial height of the cupola-shaped curvature is smaller than a radial diameter of the elastic wall portion.

10. The catheter arrangement according to claim 1, wherein the fluid passage is formed by a slot arrangement which has at least one first slot and a second slot that form at least one common intersecting point.

11. The catheter arrangement according to claim 10, wherein the slot arrangement further comprises a third slot, and wherein the at least one first slot, the second slot and the third slot are arranged in a star-shaped manner, forming form two common intersecting point.

12. The catheter arrangement according to claim 10, wherein the slot arrangement further comprises a third slot, and wherein the at least one first slot, the second slot and the third slot are arranged in a star-shaped manner, forming only one common intersecting point.

13. The catheter arrangement according to claim 10, wherein the elastic wall portion has at least one first pair of rib elements and one second pair of rib elements that are in each case arranged opposite one another in pairs with respect to the at least one common intersecting point, wherein the first pair of rib elements is arranged offset radially further outwards with respect to the intersecting point than the second pair of rib elements.

14. The catheter arrangement according to claim 1, wherein at least one further fluid passage is made to extend through the elastic wall portion, wherein the at least one further fluid passage is formed by at least two circumferential slots made to extend longitudinally in the circumferential direction of the elastic wall portion, and wherein the at least two circumferential slots are arranged offset radially outwards relative to the fluid passage.

15. The catheter arrangement according to claim 14, wherein the circumferential slots are in each case longer on a distal side of the elastic wall portion than on a proximal side of the elastic wall portion, and/or the fluid passage has at least one slot which is longer on the proximal side of the elastic wall portion than on the distal side of the elastic wall portion.

16. The catheter arrangement according to claim 14, wherein the elastic wall portion comprises a first elastic wall portion and a second elastic wall portion, wherein the first elastic wall portion has at least one recessed or raised profiling in a region of the fluid passage, and/or the second elastic wall portion has at least one recessed or raised further profiling in the region of the at least one further fluid passage.

17. The catheter arrangement according to claim 1, wherein the valve element has an encircling radial collar which is fixed in an encircling radial groove of the housing body, wherein the housing body is configured as a single piece.

18. The catheter arrangement according to claim 1, wherein the valve element has a radial collar which is fixed between two joined-together housing parts of the housing body.

19. The catheter arrangement according to claim 10, wherein the at least one first slot and the second slot are arranged in a cross-shaped manner, forming the at least one common intersecting point.

20. The catheter arrangement according to claim 11, wherein the at least one first slot, the second slot and the third slot are arranged in a H-shaped manner, forming the two common intersecting points.
